(19) United States
(12) Patent Application Publication
(10) Pub. No.: US 2019/0125433 A1
(43) Pub. Date: May 2, 2019

(54) BIPOLAR-MICROSCISSOR FORCEPS ASSEMBLY

(71) Applicant: GLOBUS MEDICAL, INC., AUDUBON, PA (US)

(72) Inventor: Richard Ashpole, Loughborough Leicestershire (GB)

(21) Appl. No.: 16/166,214

(22) Filed: Oct. 22, 2018

Related U.S. Application Data
(60) Provisional application No. 62/579,925, filed on Nov. 1, 2017.

(51) Int. Cl. A61B 18/14; A61B 18/12

(52) U.S. Cl. CPC A61B 18/1445; A61B 18/1206; A61B 2018/00172; A61B 2018/126; A61B 2018/146

(57) ABSTRACT

A bipolar-microscissor forceps assembly includes a forceps having a first forceps arm having a first forceps distal tip and a first forceps proximal end and a second forceps arm having a second forceps distal tip and a second forceps proximal end. The second forceps proximal end is connected to the first forceps proximal end. A microscissors is disposed between the first forceps arm and the second forceps arm. A kit with a plurality of microscissors releasably connected to the forceps is also provided.

### CROSS REFERENCE TO RELATED APPLICATION

This application claims priority to provisional application 62/579,925, filed Nov. 1, 2017, which is incorporated by reference herein in its entirety for all purposes.

### BACKGROUND

One of the fundamental processes in microneurosurgery is the dissection of structures from one another in order to establish a plane of cleavage between them. This is done predominantly using three instruments: a microsucker, bipolar diathermy forceps, and microscissors. The plane is developed using a pair of bipolar diathermy forceps in the dominant hand, along with a microsucker in the non-dominant hand. This results in a plane between the structures that is crossed by any number of vessels of varying sizes. These vessels, if they can be sacrificed, are “buzzed” between the tips of the bipolar forceps to leave a shriveled and occluded vessel.

The bipolar forceps are then exchanged, via the scrub nurse, for an appropriately sized and shaped pair of microscissors, which are then used to cut the vessel, before being exchanged for the bipolar forceps once more. The dissection process thus proceeds with multiple swappings between the bipolar forceps and the microscissors as the plane is developed. This thus has a rather staccato quality, especially if a microscope is involved, as the two instruments are constantly brought in and out of the magnified field. It would be beneficial to provide an instrument that would make this process more ergonomic, as well as easier and faster.

### SUMMARY

To meet this and other needs, devices, assemblies, kits, and methods are described herein, which combines bipolar forceps with microscissors in order to make dissection faster, more accurate, and/or more ergonomic for the surgeon.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, a bipolar-microscissor forceps assembly includes a forceps having a first forceps arm having a first forceps distal tip and a first forceps proximal end and a second forceps arm having a second forceps distal tip and a second forceps proximal end. The second forceps proximal end is connected to the first forceps proximal end. A microscissor is disposed between the first forceps arm and the second forceps arm.

In an alternative embodiment, a bipolar-microscissor forceps assembly comprises a bipolar forceps having a first forceps arm having a first forceps distal tip and a first forceps proximal end. A first electrical lead extends proximally from the first forceps arm. A second forceps arm has a second forceps distal tip and a second forceps proximal end. The second forceps proximal end is connected to the first forceps proximal end. A second electrical lead extends proximally from the second forceps arm. A microscissor is disposed between the first forceps arm and the second forceps arm and is electrically insulated from the forceps.

In still another alternative embodiment, a kit comprises a forceps having a first forceps arm having a first forceps distal tip and a first forceps proximal end and a second forceps arm having a second forceps distal tip and a second forceps proximal end. The second forceps proximal end is connected to the first forceps proximal end. A plurality of microscissors is provided, with each of the plurality of microscissors being releasably connected to the forceps between the first forceps arm and the second forceps arm. Each of the plurality of microscissors has a different length than the remaining of the plurality of microscissors.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of a bipolar-microscissor forceps assembly according to a first exemplary embodiment of the present invention;

FIG. 2 is a top plan view of the bipolar-microscissor forceps assembly shown in FIG. 1;

FIG. 3 is a perspective view of a bipolar-microscissor forceps assembly according to a second exemplary embodiment of the present invention;

FIG. 4 is an exploded view of the connection of the scissors and forceps shown in FIG. 3, taken along enlargement circle 4 shown in FIG. 3;

FIG. 5 is a top plan view of a bipolar-microscissor forceps assembly according to a third exemplary embodiment of the present invention;

FIG. 6 is a top plan view showing the scissors shown in FIG. 5 being slid onto the forceps shown in FIG. 5;

FIG. 7 is an enlarged view of an alternative location of a sleeve used to connect the scissors shown in FIG. 5 to the forceps shown in FIG. 5;

FIG. 8 is a top plan view of a bipolar-microscissor forceps assembly according to a fourth exemplary embodiment of the present invention;

FIG. 9 is an enlarged view showing the scissors shown in FIG. 8 being slid into the forceps shown in FIG. 8;

FIG. 10 is a perspective view of a proximal end of a bipolar-microscissor forceps assembly according to a fifth exemplary embodiment of the present invention;

FIG. 11 is a perspective view of a proximal end of a bipolar-microscissor forceps assembly according to a sixth exemplary embodiment of the present invention;

FIG. 12 is a to plan view of a proximal end of a bipolar-microscissor forceps assembly according to another exemplary embodiment of the present invention;

FIG. 13 is a top plan view of the scissor operating mechanism shown in FIG. 13;

FIG. 14 is a side elevational view of the assembly shown in FIG. 12, showing optional control surfaces for operating the scissors;

FIG. 15A is a side elevational view of the assembly shown in FIG. 14, with an operator's finger on the control surface;

FIG. 15B is a top plan view of the assembly shown in FIG. 14, with an optional cover;

FIG. 15C is a perspective view of the assembly and cover shown in FIG. 15B;

FIG. 16 is a side perspective view of an alignment block/stop that can be used with any of the embodiments of the present invention;

FIG. 17 is a front perspective view of the alignment block/stop shown in FIG. 16; and

FIG. 18 is a kit showing bipolar forceps and a plurality of scissors of differing sizes that can be used with the forceps.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term “proximal” means a direction closer to a user of the inventive instrument and “distal” means a direction farther from the user of the inventive instrument.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

The inventive instrument can be used for neurosurgical procedures including, but not limited to, dissection of meningiomata and gliomata; dissection along narrow anatomical corridors for aneurysms, acoustic neuromas, and other tumors of the cerebello-pontine angle (CPA) and skull base; dissection along interhemispheric and transcallosal routes to intraventricular structures; posterior fossa and cranio-cervical junction approaches; and microsurgical dissection in spinal neuro surgery.

While features of one or more embodiments are described with respect to a particular embodiment, those skilled in the art will recognize that features of a particular embodiment can be provided in other embodiments as well.

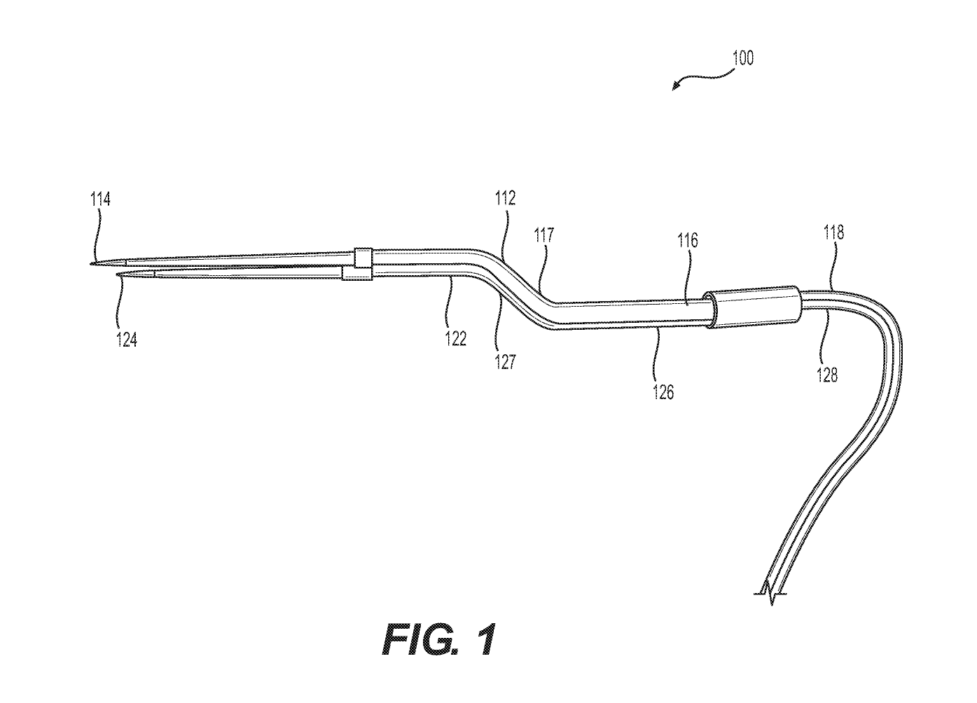

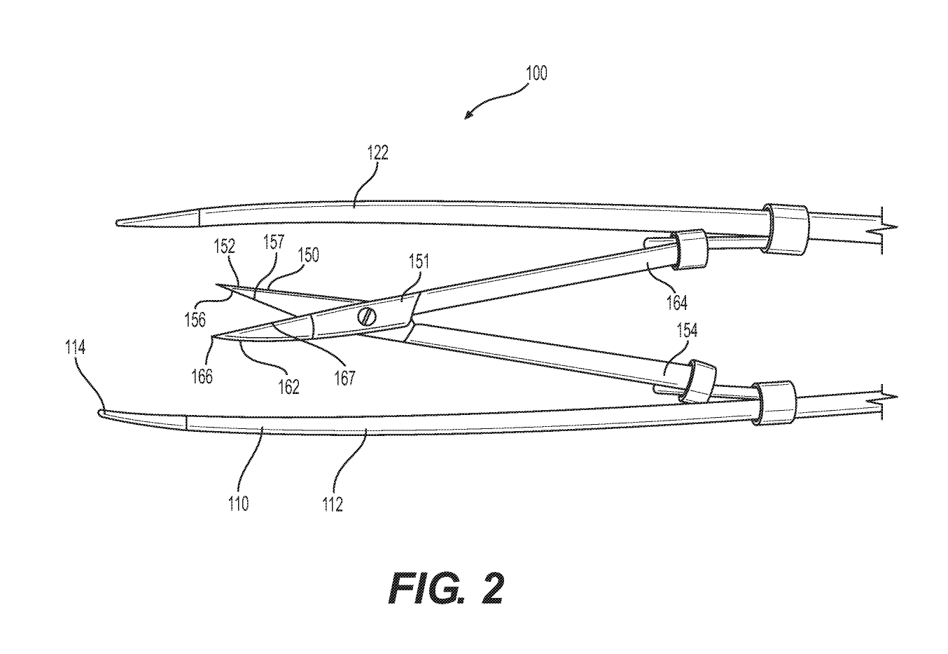

Referring to the Figures, specifically to FIGS. 1 and 2, a bipolar-microscissor forceps assembly 100 (“assembly 100”) according to a first exemplary embodiment of the present invention is shown. Assembly 100 includes a combined forceps 110 and a pair of microscissors 150. Assembly 100 eliminates the need to switch between separate forceps and scissors during microneurosurgery operations.

Forceps 110 includes a first forceps arm 112 having a first forceps distal tip 114 and a first forceps proximal end 116. First distal tip 114 is an electrically conductive metal that is electrically connected to an electrical supply (not shown) via a first electrical conducting cable 118.

Forceps 110 also includes a second forceps arm 122 having a second forceps distal tip 124 and a second forceps proximal end 126. Additionally, second forceps proximal end 126 is connected to first forceps proximal end 116 such that distal tips 114, 124 are biased away from each other and require manual force somewhere along the length of forceps arms 112, 122 to close distal tips 114, 124 toward each other.

First distal tip 114 is an electrically conductive metal that is electrically connected to an electrical supply (not shown) via a first electrical conducting cable 118 extending proximally from first forceps arm 112. Similarly, second distal tip 124 is an electrically conductive metal that is electrically connected to the electrical supply via a second electrical conducting cable 128 extending proximally from second forceps arm 122. Forceps arms 112, 122 proximal of distal tips 114, 124 are electrically insulated so that assembly 100 can be grasped without electrically shocking the user.

In an exemplary embodiment, shown in FIG. 1, forceps arms 112, 122 proximal of distal tips 114, 124 include an offset 117, 127 such that distal tips 114, 124 extend generally in a first plane and proximal ends 116, 126 extend in in a separate plane. Offsets 117, 127 are provided for ergonomic reasons to provide a better gripping surface for the user and are formed to accommodate scissors 150. Additionally, first forceps arm 112 and the second forceps arm 122 each has a superior forceps surface 119, 129, respectively.

Referring to FIG. 2, microscissors 150 are disposed between first forceps arm 112 and second forceps arm 122 and are electrically insulated from forceps 110. Microscissors 150 includes a first scissors arm 152 having a first scissors proximal end 154 connected to first forceps arm 112 and a second scissors arm 162 having a second scissors proximal end 164 connected to second forceps arm 122. In an exemplary embodiment, microscissors 150 are fixedly connected to forceps 110 so that assembly 100 is an integrated unit.

Microscissors 150 have a superior microscissors surface 151, flush with the forceps superior surfaces 119, 129. In assembly 100 as shown in FIG. 2, second scissors arm 162 is the superior scissors arm, so surface 151 is generally the top surface of second scissors arm 162. As further shown in FIG. 2, microscissors 150 are located entirely proximal of first forceps distal tip 114 and second forceps distal tip 124.

Microscissors 150 also include first and second distal tips 156, 166 having complimentary cutting blades 157, 167 that are used to cut tissue. Distal tips 156, 166 can be straight, curved, or any desired shape, depending on the anticipated use of microscissors 150. Microscissors 150 are activated by compressing forceps arms 112, 122 toward each other.

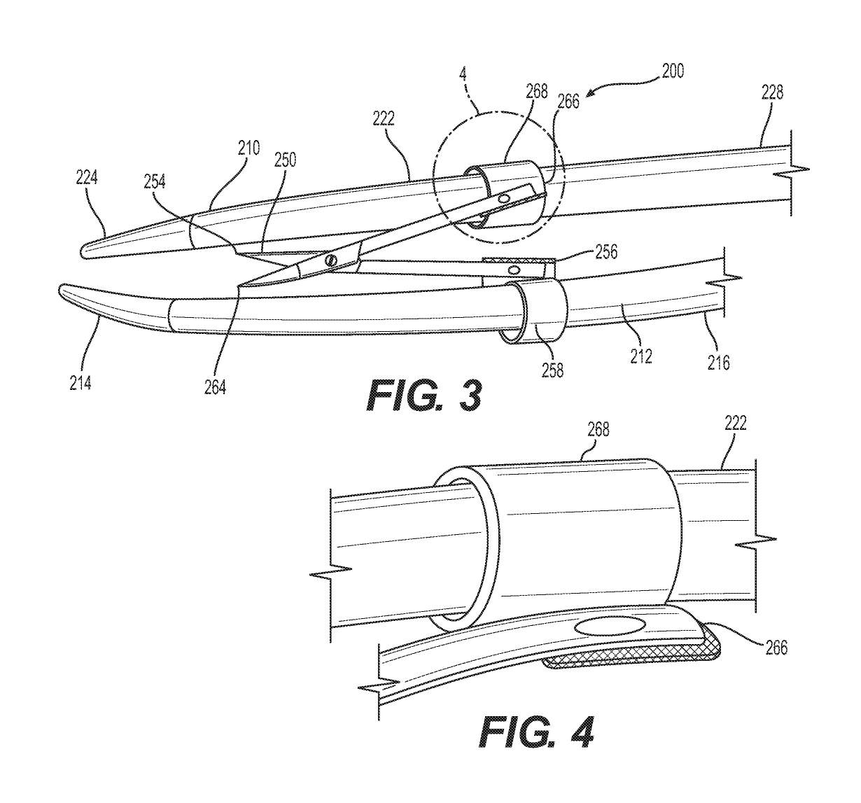

In an alternative embodiment, shown in FIGS. 3 and 4, a bipolar-microscissors forceps assembly 200 (“assembly 200”) according to a second exemplary embodiment of the present invention is shown. Assembly 200 includes forceps 210 and a scissors 250, similar to assembly 100 discussed above. Contrary to assembly 100, however, in which scissors 150 are fixed to forceps 110, scissors 250 are removably connected to forceps 210. Forceps 210 can be the same as or similar to forceps 110.

Scissors 250 has a first scissors proximal end 256 removably connected to a first forceps arm 212 and a second scissors proximal end 266 removably connected to a second forceps arm 222.

First scissors proximal end 256 includes comprises a first clip 258 that is adapted to clip onto first forceps arm 212 between a first forceps distal tip 214 and a first forceps proximal end 216. Similarly, second scissors proximal end 266 includes a second clip 268 that is adapted to clip onto second forceps arm 222 between a second forceps distal tip 224 and a second forceps proximal end 226. Clips 258, 268 can be located anywhere along the length of forceps arms 212, 222, as long as distal tips 254, 264 of scissors do not extend distally of distal tips 214, 224 of forceps 210. Clips 258, 268 have a maximum opening size that is slightly smaller than the diameter of forceps arms 212, 222 so that clips 258, 268 are biased open over and engage forceps arms 212, 222 with a firm, frictional fit.

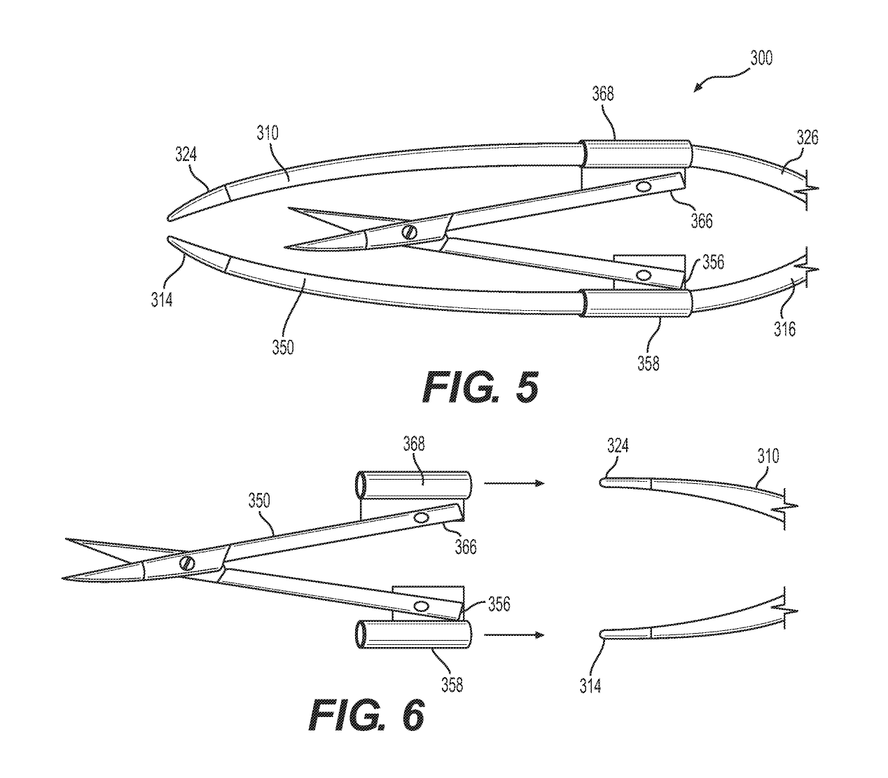

In an alternative embodiment, shown in FIGS. 5-7, a bipolar-microscissor forceps assembly 300 (“assembly 300”) according to a third exemplary embodiment of the present invention is shown. Assembly 300 includes a forceps 310 and a scissors 350, similar to assembly 200 discussed above. Forceps 310 can be the same as or similar to forceps 210.

Contrary to assembly 200, however, in which scissors 250 are removably connected to forceps 210 via clips 258, 268 that are clipped onto forceps arms 212, 222, scissors 350 include sleeves 358, 368 located at proximal ends 356, 366, respectively, that are removably slid over forceps distal tips 314, 324 from forceps distal tips 314, 324 toward proximal ends 316, 326 (shown in FIG. 6) so that sleeves 358, 368 engage forceps arms 312, 322 with a firm, frictional fit.

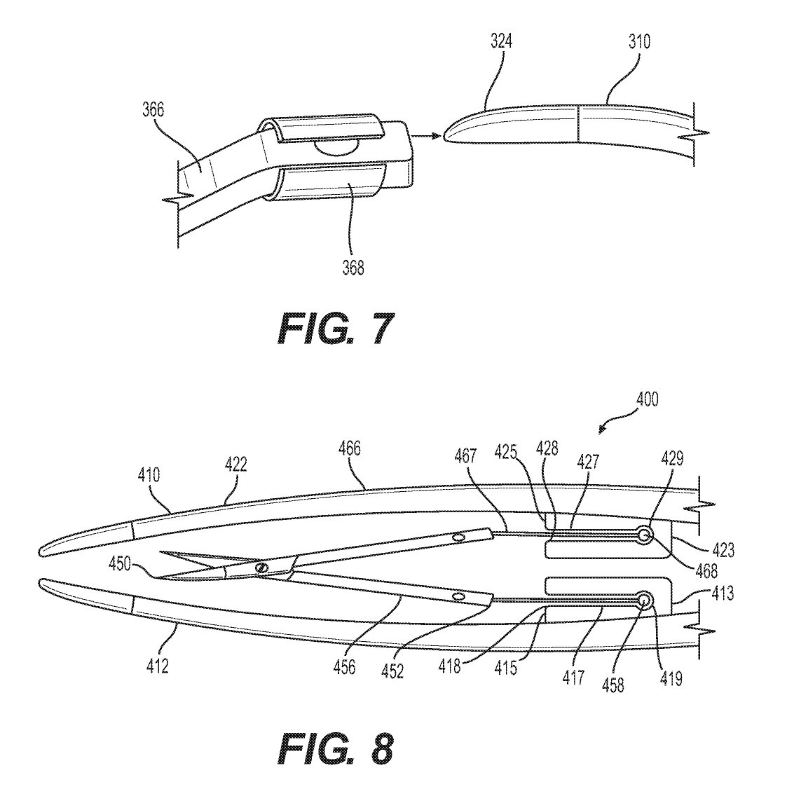

Sleeves 358, 368 are thin so that the vision of the user is uninterrupted and sleeves 358, 368 must also be a tight fit onto forceps arms 312, 322. Also, sleeves 358, 368 can be mounted on laterally with respect to their respective proximal ends 356, 366 or, alternatively, as shown in FIG. 7, below their respective proximal ends 356, 366 (only sleeve 368 and end 326 are shown in FIG. 8).

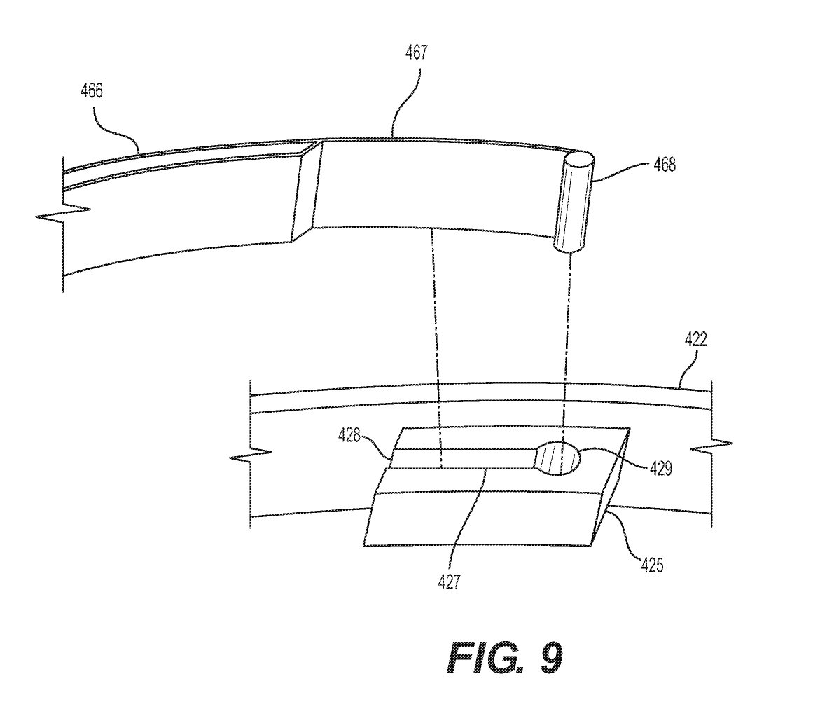

In another alternative embodiment, shown in FIGS. 8 and 9, a bipolar-microscissor forceps assembly 400 (“assembly 400”) according to a fourth exemplary embodiment of the present invention is shown. Assembly 400 includes a forceps 410 and a scissors 450, similar to assembly 300 discussed above. However, each forceps arm 412, 422 includes first and second receivers 413, 423, respectively. Receivers 413, 423 each include a ledge 415, 425 that extends inwardly of their respective forceps arms 412, 422.

Ledge 415 includes an elongate slot 417 having a distal opening 418 and a proximal terminus 419, such that terminus 419 has a larger diameter than the width of slot 417. Similarly, ledge 425 includes an elongate slot 427 having a distal opening 428 and a proximal terminus 429, such that terminus 429 has a larger diameter than the width of slot 427.

Scissors 450 includes a first scissors proximal end 456 having a first insert 457 removably insertable into slot 417 in first receiver 413. Proximal end 456 is formed from a spring material. Proximal end 456 also includes a terminal bend 458 that fits into terminus 419 so that proximal end 456 cannot slide distally from slot 417. Similarly, scissors 460 also includes a second scissors proximal end 466 having a second insert 467 removably insertable into slot 427 in second receiver 423. Proximal end 466 is formed from a spring material. Proximal end 466 also includes a terminal bend 468 that fits into terminus 429 so that proximal end 466 cannot slide distally from slot 427. As shown in FIG. 9, bend 458 is inserted into terminus 429 as insert 467 is being inserted into slot 427.

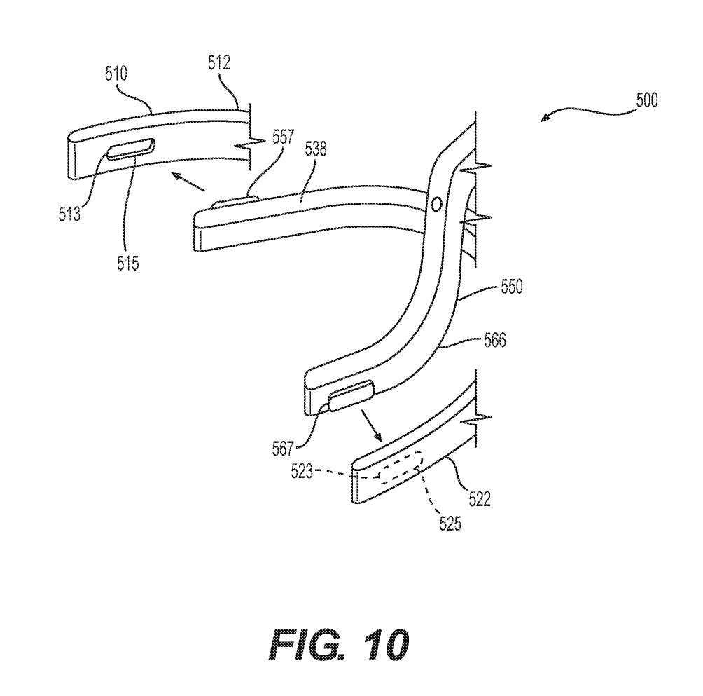

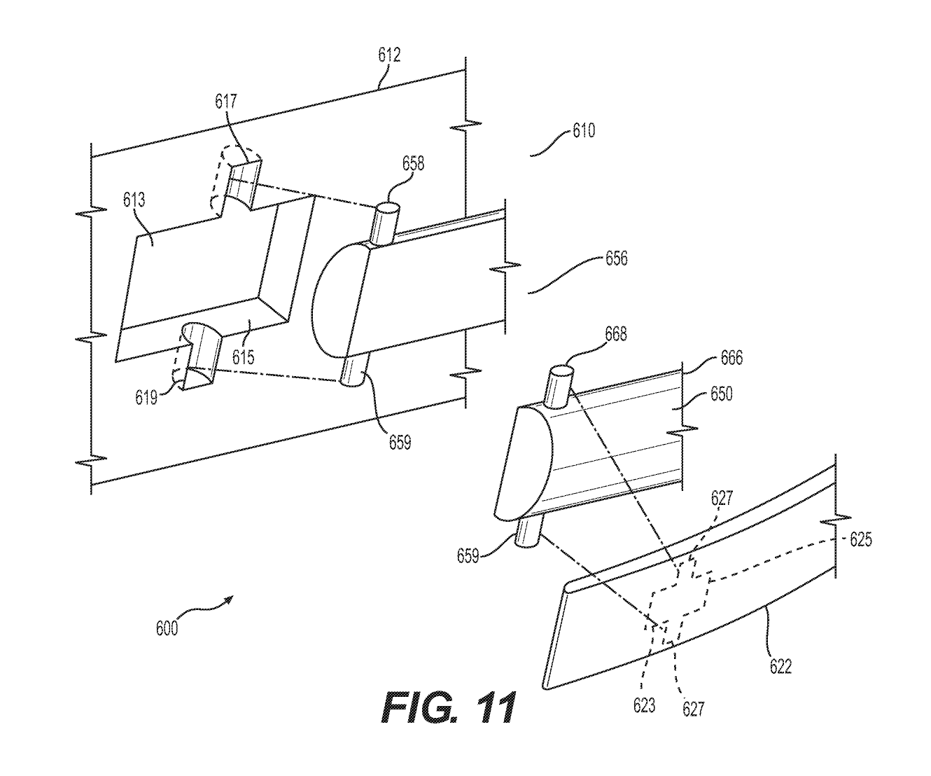

In another alternative embodiment, shown in FIG. 10, a bipolar-microscissor forceps assembly 500 (“assembly 500”) according to a fifth exemplary embodiment of the present invention is shown. Assembly 500 includes a forceps 510 and a scissors 550, similar to assembly 400 discussed above. However, each forceps arm 512, 522 includes first and second receivers 513, 523, respectively, that are built into respective forceps arms 512, 522. Receivers 513, 523 each include a slot 515, 525 that is cut into respective forceps arms 512, 522. As shown in FIG. 11, slots 515, 525 are generally oblong in shape.

Scissors 550 includes first and second scissors proximal ends 556, 566 that each include a nub 557, 567 extending outwardly from respective scissor arms 512, 522. Nubs 557, 567 are sized and shaped for a tight frictional fit within slots 515, 525, respectively, such that microscissors 550 are releasably retained within forceps 510, but will not easily be dislodged from forceps 510. Optionally, a spring (not shown) can be located between proximal ends 556, 566 to bias proximal ends 556, 566 into their respective slots 515, 525.

In another alternative embodiment, shown in FIG. 11, a bipolar-microscissor forceps assembly 600 (“assembly 600”) according to a sixth exemplary embodiment of the present invention is shown. Assembly 600 includes a forceps 610 and a scissors 650, similar to assembly 500 discussed above. However, each forceps arm 612, 622 includes first and second receivers 613, 623, respectively, that are built into respective forceps arms 612, 622. Receivers 613, 623 each include a slot 615, 625 that is cut into respective forceps arms 612, 622. As shown in FIG. 11, slots 615, 625 are generally rectangular in shape and further include superior and inferior pin retaining slots 617, 619 and 627, 629, respectively.

Scissors 650 includes first and second scissors proximal ends 656, 666 that each include superior and inferior pins 658, 659 and 668, 669 extending upwardly/downwardly from respective scissor arms 612, 622 such that pins 658, 659 fit into pin retaining slots 617, 619 and pins 668, 669 fit into pin retaining slots 627, 629. Pins 658, 659, 668, 669 pivot in their respective pin retaining slots 617, 619, 627, 629 to allow scissors 650 to open and close.

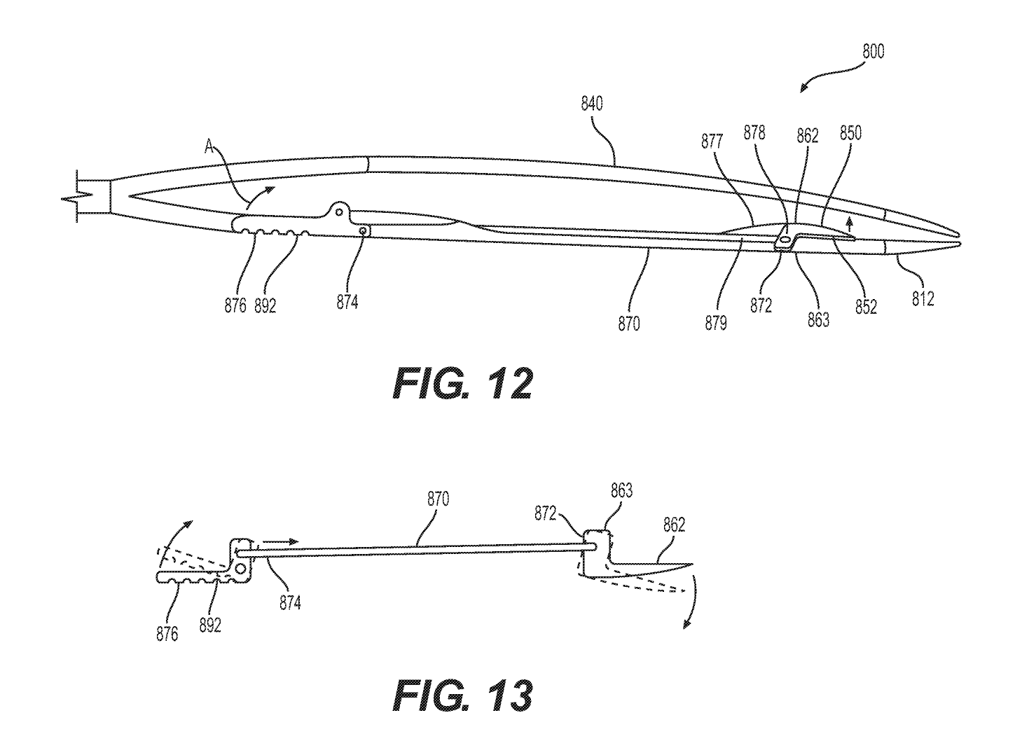

In another alternative embodiment, shown in FIGS. 12-14, a bipolar-microscissor forceps assembly 800 (“assembly 800”) according to another exemplary embodiment of the present invention is shown. Assembly 800 includes a forceps 810 and a scissors 850, with scissors 850 having a first blade 852 fixed to a first forceps arm 812 and a second blade 862 pivotally connected to one of first forceps arm 812 and first blade 852 at a pivot 863 to operate between a blade engaged position and a blade disengaged position.

A proximal scissors actuating rod 870 extends along first forceps arm 812 such that actuating rod 870 has a distal end 872 pivotally connected to second blade 862. Scissors actuating rod 870 also has a proximal actuating rod end 874 that has a lever 876 pivotally mounted to first forceps arm 812 such that pivoting of lever 876 away from first forceps arm 812 pivots second blade 862 to an open position relative to first blade 852. The dashed lines show the position of lever 876 to open second blade 862 relative to first blade 852, while the solid lines show the position of lever 876 to close second blade 862 relative to first blade 852.

Referring to FIG. 12, a biasing member 877 biases second blade 862 toward the closed position against first blade 852. In an exemplary embodiment, biasing member 877 is a leaf spring having a distal end 878 connected to second blade 862 proximate to distal end 872 of actuating rod 870 and a proximal end 879 connected to first forceps arm 812.

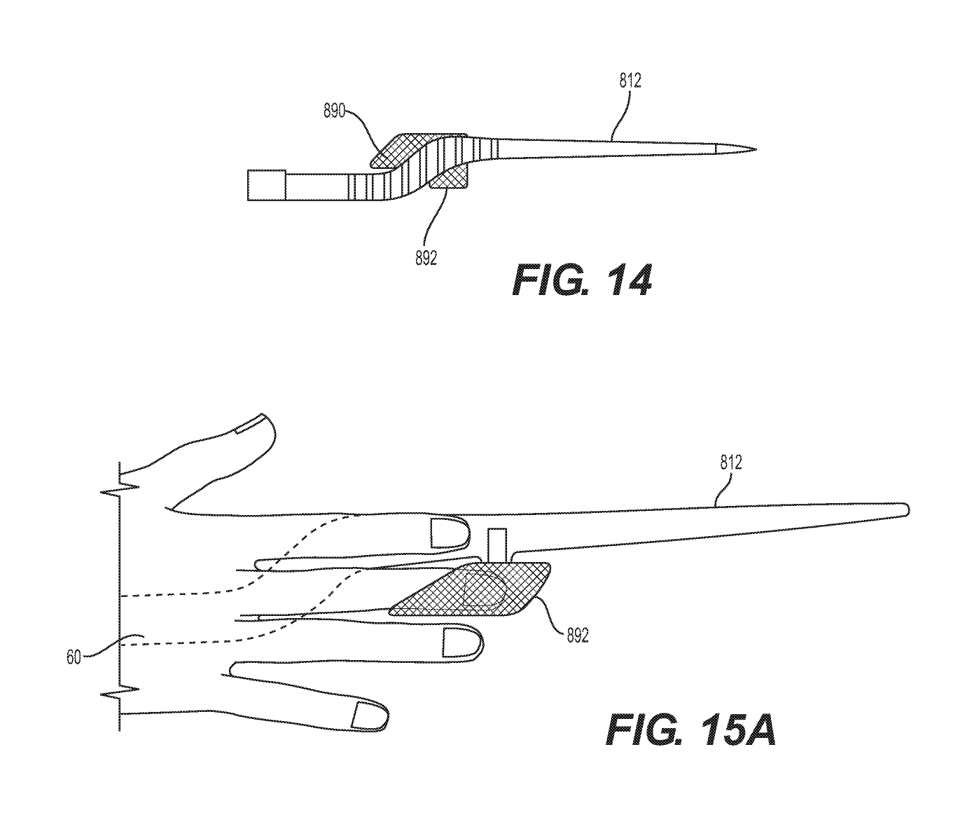

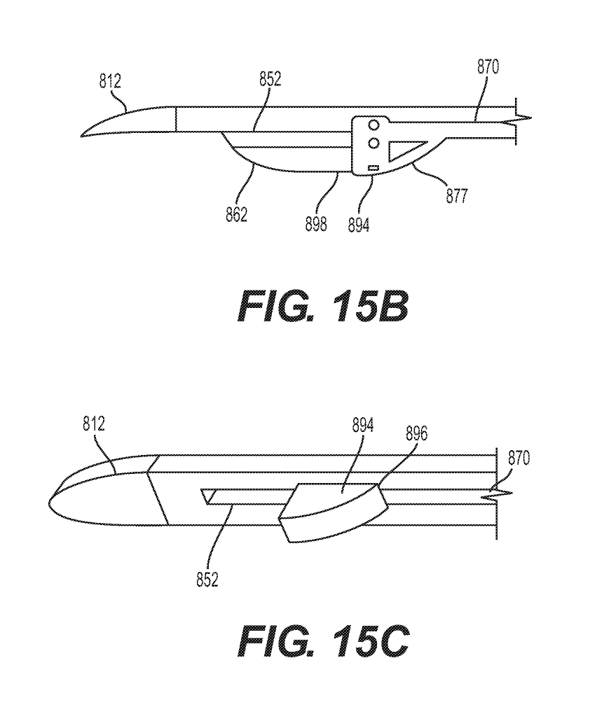

Referring to FIGS. 14 and 15A, control surfaces 890, 892 can be alternatively provided either above or below first forceps arm 812 to allow the surgeon additional control over the operation of scissors 850. Control surfaces 890, 892 are directly coupled to lever 876 such that the pushing of the selected control surface 890, 892 into the plane of the paper of FIG. 14 pushes lever 876 in the direction of arrow “A” in FIG. 12. An advantage to using control surface 892, as shown in FIG. 15A, is that a user's middle finger 60 can activate control surface 892 and, by extension, scissors 850, merely by pushing control surface 892 into the plane of the paper of FIG. 5 or 15A.

Referring to FIGS. 15B and 15C (second blade 862 omitted in FIG. 15C for clarity), a cover 894 can be optionally added over distal end 872 of actuating rod 870 at pivot 863 and biasing member 877 to protect pivot 863 and biasing member 877. Cover 894 includes a proximal opening 896 that is sized to allow for smooth operation of actuating rod 870. Similarly, cover 894 includes a distal opening 898 to allow for the pivoting action of second blade 862.

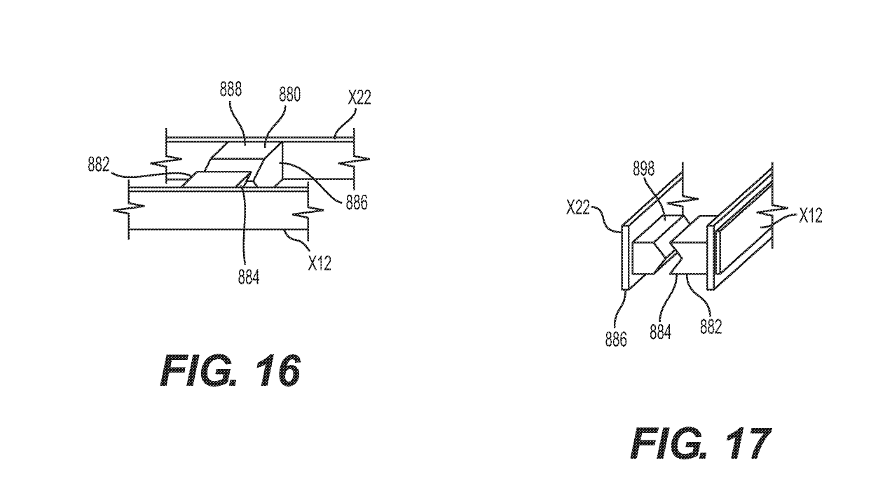

FIGS. 16 and 17 show an alignment block/stop 880 that can optionally be positioned between the forceps arms x12, x22 (where “x” is a whole number between 1 and 8) of any of the embodiments described above, generally proximally of a respective scissors, to ensure that the forceps tips align perfectly and do not cross or miss each other. Stop 880 includes a first block 882 having a generally centrally cut chamfer 884 that fits into a corresponding groove 886 in a second block 888. Alignment block/stop 880 can be used in any of the embodiments described above. Blocks 882, 888 can optionally be combined with ledges 415, 425 in assembly 400, thus combining the concept of the alignment block/stop 880 with the retainer for scissors proximal ends 456, 466 as described above.

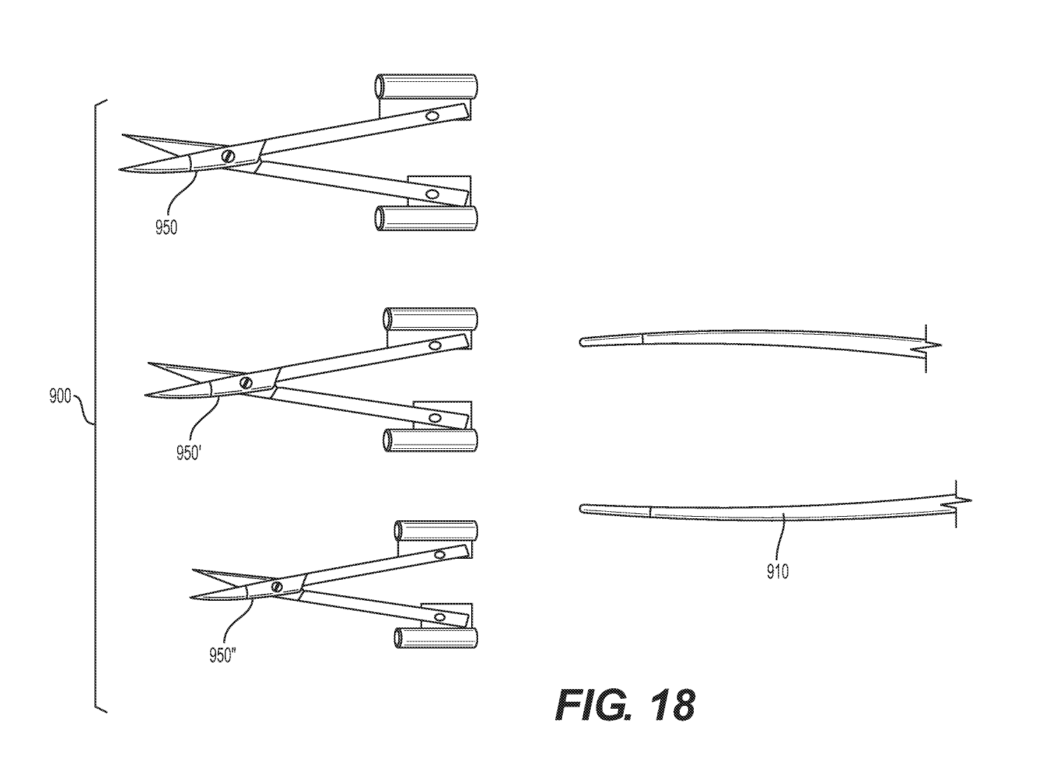

Referring now to FIG. 18, a kit 900 that includes forceps 910 with a plurality of microscissors 950, 950′, 950″ can be provided, such that each of the plurality of microscissors 950, 950′, 950″ is releasably connected to forceps 910 between the forceps arm 912 and second forceps arm 922. Microscissors 950, 950′, 950″ can be connected to forceps 910 via any of the mechanisms disclosed above, or other suitable mechanisms.

Each of the plurality of microscissors 950, 950′, 950″ has a different length than the remaining of the plurality of microscissors 950, 950′, 950″. Alternatively or additionally, each of the plurality of microscissors 950, 950′, 950″ can have different curvatures.

Further contemplated are methods of using the bipolar-microscissor forceps assembly including performing microsurgery, for example, by retracting tissue and/or dissecting tissue and the like to access a surgical site.

It will be further understood that various changes in the details, materials, and arrangements of the parts which have been described and illustrated in order to explain the nature of this invention may be made by those skilled in the art without departing from the scope of the invention as expressed in the following claims.

I claim:

1. A bipolar-microscissor forceps assembly comprising:
a forceps comprising:
a first forceps arm having a first forceps distal tip and a first forceps proximal end; and
a second forceps arm having a second forceps distal tip and a second forceps proximal end, the second forceps proximal end being connected to the first forceps proximal end; and
a microscissors disposed between the first forceps arm and the second forceps arm.

2. The bipolar-microscissor forceps assembly according to claim 1, wherein the microscissors comprises:
a first scissors arm having a first scissors proximal end connected to the first forceps arm; and
a second scissors arm having a second scissors proximal end connected to the second forceps arm.

3. The bipolar-microscissor forceps assembly according to claim 2,
wherein the first scissors proximal end is removably connected to the first forceps arm; and
wherein the second scissors proximal end is removably connected to the second forceps arm.

4. The bipolar-microscissor forceps assembly according to claim 2,
wherein the first scissors proximal end comprises a first clip adapted to clip onto the first forceps arm between the first forceps distal tip and the first forceps proximal end; and
wherein the second scissors proximal end comprises a second clip adapted to clip onto the second forceps arm between the second forceps distal tip and the second forceps proximal end.

5. The bipolar-microscissor forceps assembly according to claim 2,
wherein the first scissors proximal end comprises a first sleeve adapted to slide over the first forceps arm from the first forceps distal tip toward the first forceps proximal end; and
wherein the second scissors proximal end comprises a second sleeve adapted to slide over the second forceps arm from the second forceps distal tip toward the second forceps proximal end.

6. The bipolar-microscissor forceps assembly according to claim 2,
wherein the first forceps arm comprises a first receiver and wherein the first scissors proximal end comprises a first insert removably insertable into the first receiver; and
wherein the second forceps arm comprises a second receiver and wherein the second scissors proximal end comprises a second insert removably insertable into the second receiver.

7. The bipolar-microscissor forceps assembly according to claim 1, wherein the microscissors comprises a first blade fixed to the first forceps arm and a second blade pivotally connected to one of the first forceps arm and the first blade.

8. The bipolar-microscissor forceps assembly according to claim 7, further comprising an actuating rod extending along the first forceps arm, the actuating rod having a distal end connected to the second blade.

9. The bipolar-microscissor forceps assembly according to claim 8, wherein the scissors actuating rod has a proximal actuating rod end having a lever pivotally mounted to the first forceps arm such that pivoting of the lever away from the first forceps arm pivots the second blade to an open position relative to the first blade.

10. The bipolar-microscissor forceps assembly according to claim 1, wherein the microscissors are electrically insulated from the forceps.

11. The bipolar-microscissor forceps assembly according to claim 1, wherein the first forceps arm and the second forceps arm each has a superior forceps surface and wherein the microscissors has a superior microscissors surface, flush with the forceps superior surface.

12. A bipolar-microscissor forceps assembly comprising:
a bipolar forceps comprising:
a first forceps arm having a first forceps distal tip and a first forceps proximal end and a first electrical lead extending proximally from the first forceps arm; and
a second forceps arm having a second forceps distal tip and a second forceps proximal end, the second forceps proximal end being connected to the first forceps proximal end, and a second electrical lead extending proximally from the second forceps arm; and
a microscissors disposed between the first forceps arm and the second forceps arm and being electrically insulated from the forceps.

13. The bipolar-microscissor forceps assembly according to claim 12, wherein the microscissors are located entirely proximal of the first forceps distal tip and the second forceps distal tip.

14. The bipolar-microscissor forceps assembly according to claim 12, wherein the microscissors comprises a first blade attached to the first forceps arm and a second blade attached to the assembly to operate between a blade engaged position and a blade disengaged position.

15. The bipolar-microscissor forceps assembly according to claim 14, further comprising a proximal actuating rod connected to the second blade.

16. The bipolar-microscissor forceps assembly according to claim 12, wherein the microscissors are removably connected to the forceps.

17. The bipolar-microscissor forceps assembly according to claim 16, wherein the first forceps arm comprises receiver adapted to removably retain the microscissors therein.

18. The bipolar-microscissor forceps assembly according to claim 17, wherein receiver comprises a ledge extending toward the second forceps arm, wherein the ledge comprises a slot formed therein.

19. The bipolar-microscissor forceps assembly according to claim 18, wherein the microscissors comprises a first microscissors arm having a proximal end formed from a spring material, and wherein a distal end is removably inserted into the slot.

20. A kit comprising:
a forceps comprising:
a first forceps arm having a first forceps distal tip and a first forceps proximal end; and
a second forceps arm having a second forceps distal tip and a second forceps proximal end, the second forceps proximal end being connected to the first forceps proximal end; and
a plurality of microscissors, each of the plurality of microscissors releasably connected to the forceps between the first forceps arm and the second forceps arm, wherein each of the plurality of microscissors has a different length than the remaining of the plurality of microscissors.
